(19) United States
(12) Patent Application Publication
(10) Pub. No.: US 2016/0081669 A1
(43) Pub. Date: Mar. 24, 2016

(54) MEDICAL SPECIMEN COLLECTION SYSTEM

(71) Applicant: Timothy LOWNEY, JR., Westwood, MA (US)

(72) Inventor: Timothy LOWNEY, JR., Westwood, MA (US)

(21) Appl. No.: 14/859,981

(22) Filed: Sep. 21, 2015

Related U.S. Application Data
(60) Provisional application No. 62/052,766, filed on Sep. 19, 2014.

(51) Int. Cl. A61B 10/00

(52) U.S. Cl. CPC A61B 10/007; A61B 10/0058

(57) ABSTRACT

Systems for collecting medical specimens are generally described. The systems may be configured to, among other things, improve the ease of specimen collection and increase the ability of a patient to provide the highest quality sample with no loss of specimen. A system may include a collection container and a specimen collection portion removably coupled to the collection container. A system may be configured to include a base portion and a specimen collection portion extending from the base portion, the base portion and the specimen collection portion being a single monolithic piece and arranged to form a substantially bent cylinder shape. A system may include a base portion and a specimen collection portion extending from the base portion formed from a single monolithic piece in which the base portion has a substantially cylindrical shape and the specimen collection portion has a substantially funnel shape.

### CROSS-REFERENCE TO RELATED APPLICATIONS

This application claims priority to U.S. Provisional Application No. 62/052,766, filed Sep. 19, 2014 entitled “Medical Specimen Collection System,” the contents of which are incorporated herein by reference in their entirety.

### FIELD OF INVENTION

The described technology generally relates to medical specimen collection systems, and, more specifically, to medical specimen collection systems that include collection devices configured for the sterile, hygienic, and effective collection of urine and semen samples.

### BACKGROUND

The collection of adequate and sterile medical specimens is critical to achieving meaningful and accurate test results. However, patients obtaining medical specimens using conventional methods and collection devices face multiple challenges that prevent them from providing medical professionals with fully adequate and sterile specimens. For example, patients may experience difficulty providing urine samples without contaminating the samples due to contact between the patient and the collection container and/or sample, particularly due to the proximity required between the patient and the container during the collection process. In another example, male patients experience difficulty when providing semen samples due to the problematic angle and alignment that is required with a conventional collection container. As such, the collection of medical specimens using conventional techniques and collection devices results in the loss of medical specimen volume and/or contamination of the medical specimen. Accordingly, patients and medical professionals would benefit from a medical specimen collection system that facilitates a more sterile and effective process for collecting medical specimens.

### SUMMARY

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

In an embodiment, a medical specimen collection system may include a collection container and a specimen collection portion in fluid communication with the collection container, the specimen collection portion being configured to receive a fluid medical specimen from a patient and to facilitate a flow of the fluid medical into the specimen container, wherein the specimen collection portion has a width of about 5 centimeters to about 10 centimeters and a height of about 5 centimeters to about 10 centimeters.

In an embodiment, a medical specimen collection system may include a base portion and a specimen collection portion extending from the base portion, the base portion and the specimen collection portion being a single monolithic piece and arranged to form a substantially bent cylinder shape, wherein at least a portion of the specimen collection portion has a first diameter greater than a second diameter of the base portion and comprises an opening having a substantially elliptical shape, wherein the base portion has a width of about 6 centimeters to about 10 centimeters, the opening has a diameter of about 6 centimeters to about 10 centimeters, and the medical specimen collection system has a height of about 8 centimeters to about 20 centimeters.

In an embodiment, a medical specimen collection system may include a base portion and a specimen collection portion extending from the base portion, the base portion and the specimen collection portion being a single monolithic piece, wherein the base portion has a substantially cylindrical shape and the specimen collection portion has a substantially funnel shape with a narrow end of the specimen collection portion extending from an upper portion of the base portion, wherein an upper portion of the specimen collection portion forms an opening having a first diameter greater than a second diameter of the base portion and comprises an opening having a substantially circular shape, wherein the base portion has a width of about 6 centimeters to about 8 centimeters, the opening has a diameter of about 10 centimeters to about 15 centimeters, and the medical specimen collection system has a height of about 8 centimeters to about 20 centimeters.

### BRIEF DESCRIPTION OF THE DRAWINGS

The above and other objects of the present invention will become more readily apparent from the following detailed description taken in connection with the accompanying drawings.

FIGS. 1A-1C depict an illustrative collection system according to a first embodiment.

FIGS. 2A and 2B depict an illustrative collection system according to a second embodiment.

FIGS. 3A and 3B depict an illustrative collection system according to a third embodiment.

### DETAILED DESCRIPTION

The described technology generally relates to a system for collecting medical specimens. In particular, some embodiments provide a medical specimen collection system (the “collection system”) configured for the collection of fluid medical specimens (or “medical specimens”). The collection system, among other things, improves the ease of specimen collection and increases the ability of a patient to provide the highest quality sample with no loss of specimen.

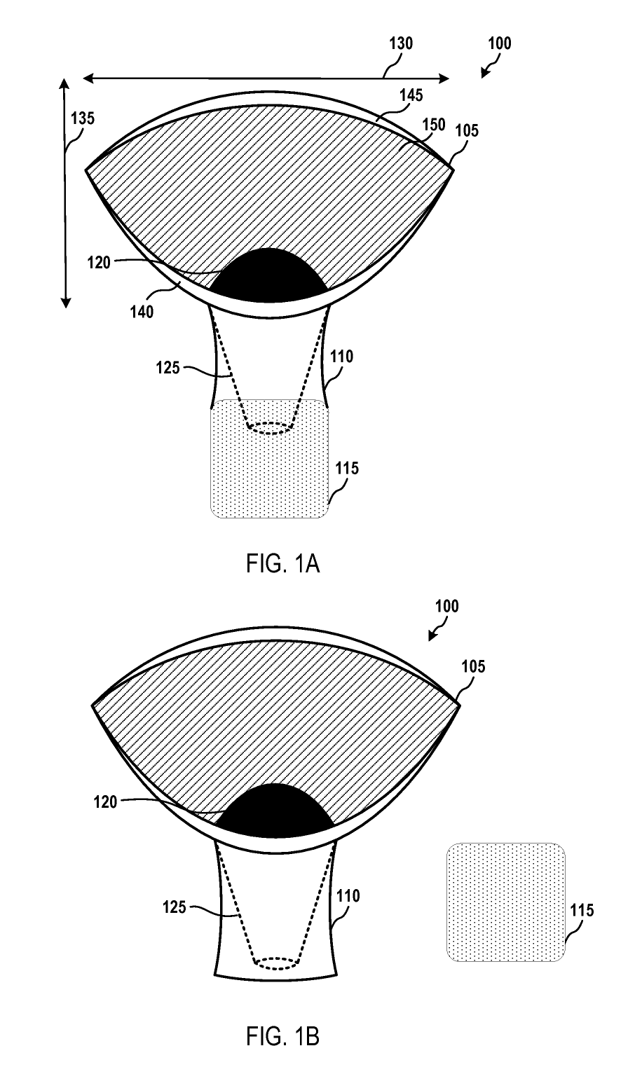

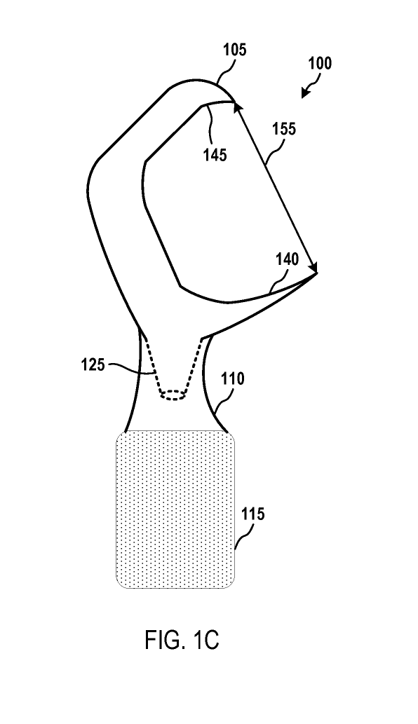

FIGS. 1A-C depict an illustrative collection system according to a first embodiment. FIGS 1A and 1B show a front view of the collection system and FIG. 1C depicts a side view of the collection system. As shown in FIGS. 1A-C, the collection system 100 may include a specimen collection portion 105 configured to receive a medical specimen from the patient. Non-limiting examples of medical specimens include semen and urine. The specimen collection portion 105 may include a lower portion 140, an upper portion 145, and an inner surface 150.

In some embodiments, the specimen collection portion 105 may have a size and shape configured to provide a patient with a sufficiently large surface area for depositing the sample that alleviates issues with spilling the sample, missing the specimen container, and/or contaminating the sample. The specimen collection portion 105 and the opening portion thereof may have various shapes including, without limitation elliptical, circular, mouth-shaped, or any combination thereof.

In some embodiments the specimen collection portion 105 may have a width 130 of about 5 centimeters, about 10 centimeters, about 15 centimeters, about 20 centimeters, or any range or value between any two of these values (including endpoints). In some embodiments the specimen collection portion 105 may have a height 135 of about 2 centimeters, about 5 centimeters, about 10 centimeters, about 15 centimeters, or any range or value between any two of these values (including endpoints). In some embodiments, the specimen collection portion 105 may have a convexity (measure of curvature) of about 1 centimeter, about 2 centimeters, about 3 centimeters, about 5 centimeters, about 7 centimeters, about 12 centimeters, and any value and or range between any two of these values (including endpoints). In some embodiments, the specimen collection portion 105 may have a width 130 of about 12.7 centimeters (about 5 inches), a height 135 of about 10.2 centimeters (about 4 inches), and a convexity of about 3.81 centimeters (about 1.5 inches) in depth, for example, to a posterior edge length of about 16.5 centimeters (about 6.5 inches). In some embodiments the inner surface 150 may have an area of about 2 centimeters², about 5 centimeters², about 10 centimeters², about 15 centimeters², about 20 centimeters², about 30 centimeters², about 50 centimeters², about 100 centimeters², or any range or value between any two of these values (including endpoints). In some embodiments, a distance 155 between the lower portion 140 and the upper portion 150 of the specimen collection portion 105 may be about 2 centimeters, about 5 centimeters, about 10 centimeters, about 15 centimeters, or any range or value between any two of these values (including endpoints).

The specimen collection portion 105 may be configured to receive a medical specimen, such as semen through the ejaculation process, and to facilitate the flow of the medical specimen into a collection container 115 in fluid communication with the specimen collection portion 100. The specimen collection portion 105 may have an opening 120 that allows the medical specimen to flow out of the specimen collection portion 105 and toward the collection container 115. In some embodiments, the opening 120 may be in fluid communication with a conduit 125 (for example, a spout, a funnel, or the like) that directs the flow of the medical specimen into the collection container 115. In some embodiments, the conduit 125 may be tapered and/or may have or may substantially have a funnel shape, for instance, in which the narrow end is facing the collection container 115. In some embodiments, at least a portion of the conduit 125 may be arranged within the collection container 115. In some embodiments, the conduit may have a diameter of about 2.5 centimeters (about 1 inches), about 5 centimeters (about 2 inches), about 7.5 centimeters (about 3 inches), about 10 centimeters (about 4 inches), about 12.5 centimeters (about 5 inches), and any value or range between any two of these values (including endpoints).

The specimen collection portion 105 is not limited to being connected to or facilitating the flow of a medical specimen to a collection container 115 as the specimen collection portion 105 may be used in connection with any type of collection or analysis device known to those having ordinary skill in the art.

The specimen collection portion 100 may be coupled to the collection container 115. In some embodiments, the specimen collection portion 105 may have a neck portion 110 configured to engage the collection container 115 and to couple the collection container 115 to the specimen collection portion 105. In some embodiments, the specimen collection portion 105 (e.g., through the neck portion 110 thereof) may be configured to be attach to conventional collection containers 115. In this manner, a specimen collection portion 105 may be configured according to some embodiments to be used with existing containers (e.g., standard sterile specimen collection containers). The specimen collection portion 105 may be coupled to the collection container 115 using various techniques known to those having ordinary skill in the art, such as through a friction fit, a threaded fit, a clamp, snapping elements, hooks, clips, or the like.

In some embodiments, the neck portion 110 may be configured as a handle to facilitate ease of use by a patient or medical professional handling the collection system 100 or the specimen collection portion 105. For example, a patient providing a semen sample may hold the collection system 100 in one hand while the other hand is used to produce the specimen.

In some embodiments, the specimen collection portion 105, the conduit 125, and/or the neck portion 110 may be formed as a single piece, for example, one monolithic piece. In some embodiments, the specimen collection portion 105, the conduit 125, and/or the neck portion 110 may be flexible, for example, so that a patient may flex portions of the collection system 100 into a desired alternative position or configuration. In some embodiments, the collection system 100 may maintain the alternative position. In some embodiments, the collection system 100 may move back to an original position responsive to removal of the force flexing the portions of the collection system.

In some embodiments, the specimen collection portion 105 and/or portions thereof may be formed from biocompatible materials, for example, materials that will not react with medical specimens. In some embodiments, the specimen collection portion 105 and/or portions thereof may be formed from materials configured to facilitate the flow or “sliding” of medical specimens from the inner surface of the specimen collection portion 105 toward the collection container 115. Non-limiting examples of materials may include a plastic material, a metal material, a polystyrene material, a polyethylene material, a latex material, a rubber material, a polytetrafluoroethylene (PTFE) material (e.g., Teflon®), and any combination thereof. In some embodiments, at least a portion of the collection system 100 may be coated with a material configured to facilitate the flow or “sliding” of medical specimens, such as PTFE.

In some embodiments, the collection system 100 may include one or more closure elements (not shown) configured to seal or otherwise cover the opening 120 or the cavity of the specimen collection portion 105. In this manner, a patient may provide a medical specimen and transport the entire collection system 100 to, for example, a medical professional without having to remove the specimen collection portion 105 from the collection container 115. In some embodiments, the specimen collection portion 105, the conduit 125, and/or the neck portion 110 may be removed from the collection system and sealed using a corresponding sealing element (e.g., cap, lid, or the like).

In some embodiments, the collection system 100 may include one or more volume loss prevention elements (not shown) configured to prevent the loss of volume of the medical specimen and/or facilitate the maximum amount of the medical specimen into the collection container 115. In some embodiments, a volume loss prevention element may include a scraper, wiper, squeegee, or other device configured to direct or push the medical specimen into the collection container 115. For example, a squeegee-like element may be configured to push portions of the medical specimen located on the specimen collection portion 105 into the collection container 115. In some embodiments, a volume loss prevention element may include a removable layer or coating (not shown) arranged on a portion of the collection system 100 that may receive a portion of the medical specimen and may be removed and used to direct the medical specimen arranged thereon into the collection container 115. For instance, the specimen collection portion 105 may include a layer of cellophane or similar material on a surface that may receive the medical specimen. The cellophane layer may be at least partially removed to direct any of the medical specimen into the collection container 115. In this manner, volume of the medical specimen that may otherwise be wasted may be directed into the collection container 115 and used for analysis.

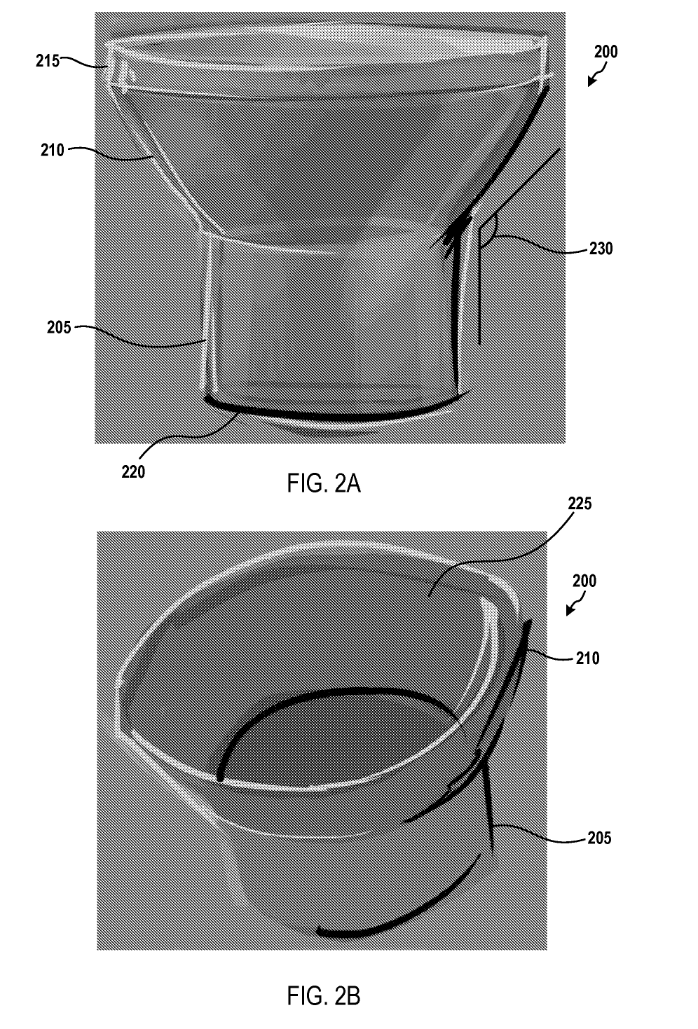

FIGS. 2A and 2B depict an illustrative collection system according to a second embodiment. FIG. 2A depicts a front view of a collection system 200 comprising a base portion 205 and a specimen collection portion 210. As shown in FIG. 2B, the specimen collection portion 210 may include an opening and an inner surface 225 configured to receive a specimen from a patient. In some embodiments, the base portion 205 may include a bottom 220 that may be coupled to a collection container, including, without limitation, a conventional collection container. For example, the bottom 220 may be coupled to a collection container through a threaded fit (i.e., an inner portion of the base portion 205 may include a threaded surface configured to correspond with a threaded portion of the collection container), a friction fit (e.g., the bottom fits tightly within the collection container, the bottom fits tightly around the collection container, or the like), or the like.

In some embodiments, the collection system 200 may include one or more closure elements 215 configured to seal or otherwise cover the opening of the specimen collection portion 210. In some embodiments, the closure element 215 may be configured as a cap 215 formed to be coupled to collection system, for example, to the specimen collection portion 210 thereof. The cap 215 may be coupled to the collection system 200 through a snap fit, a threaded (screw) fit, or any other type of coupling mechanism known to those having ordinary skill in the art.

In some embodiments, the opening of the specimen collection portion 210 may have a generally circular shape or a generally elliptical shape. In some embodiments, the base portion 205 and the specimen collection portion 210 may be formed as a single piece, for example, as a single monolithic structure. In some embodiments, the base portion 205 and/or the specimen collection portion 210 may be flexible, for example, so that a patient may flex portions of the collection system 200 into a desired position.

As shown in FIGS. 2A and 2B, the base portion 205 may have a substantially cylindrical shape and the specimen collection portion 210 may have a substantially cone or funnel shape, with the specimen collection portion extending from an upper portion of the base portion. In some embodiments, the specimen collection portion 210 may become wider as the specimen collection portion extends from the base portion 205.

In some embodiments the base portion 205 may have a width of about 3 centimeters, about 4 centimeters, about 5 centimeters, about 6 centimeters, about 8 centimeters, about 10 centimeters, or any range or value between any two of these values (including endpoints). In some embodiments, the base portion 205 may have a height of about 3 centimeters, about 4 centimeters, about 5 centimeters, about 6 centimeters, about 8 centimeters, about 10 centimeters, or any range or value between any two of these values (including endpoints). In some embodiments the specimen collection portion 210 may have a width of about 3 centimeters, about 4 centimeters, about 5 centimeters, about 6 centimeters, about 8 centimeters, about 10 centimeters, about 20 centimeters, or any range or value between any two of these values (including endpoints). In some embodiments the specimen collection portion 210 may have a height of about 3 centimeters, about 4 centimeters, about 5 centimeters, about 6 centimeters, about 8 centimeters, about 10 centimeters, or any range or value between any two of these values (including endpoints). In some embodiments, an angle 230 formed between the base portion 205 and the specimen collection portion 210 may be about 90°, about 100°, about 110°, about 120°, about 130°, about 140°, about 150°, about 160°, about 170°, or any range or value between any two of these values (including endpoints).

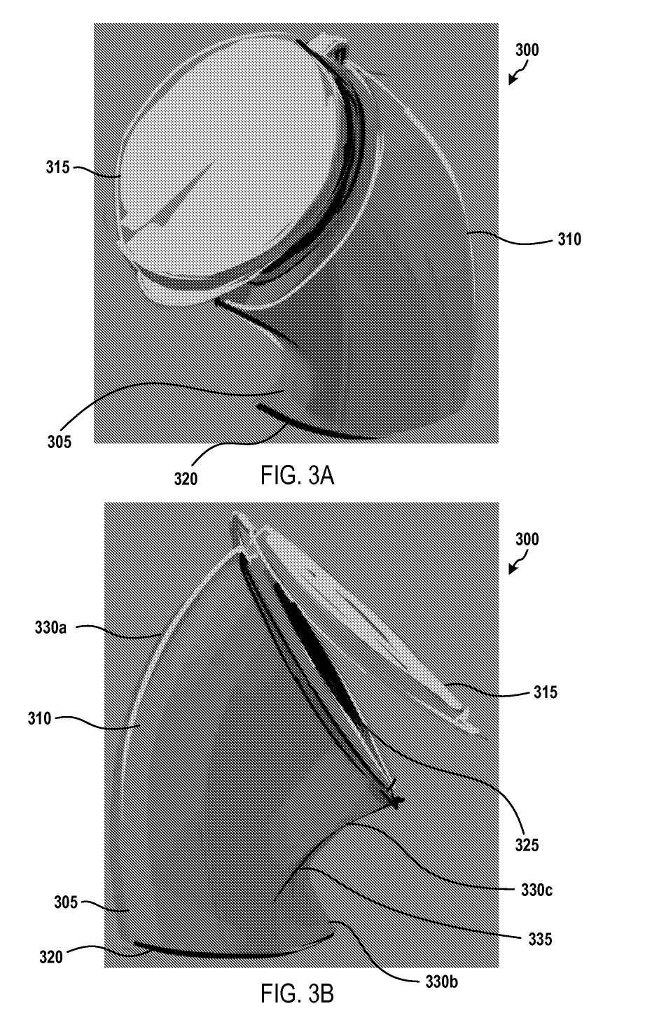

FIGS. 3A and 3B depict an illustrative collection system according to a third embodiment. As shown in FIG. 3A, a specimen collection system 300 may include a base portion 305 and a specimen collection portion 310. In some embodiments, the base portion 305 and the specimen collection portion 310 may be formed as a single piece, for example, as a single monolithic structure. In some embodiments, the specimen collection portion 310 may include an opening 325 that may have a substantially circular or elliptical shape. In some embodiments, the base portion 305 and/or the specimen collection portion 310 may be flexible, for example, so that a patient may flex portions of the collection system 300 into a desired position.

In some embodiments, the specimen collection system 300 may have a shape of or substantially of a bent or curved cylinder. In some embodiments, the specimen collection system 300 may include a convex curved portion 330a extending or substantially extending from the top of the specimen collection portion 310 to the bottom of the base portion 305. The specimen collection system 300 may include a first concave curved portion 330b and a second concave curved portion 330c arranged opposite or substantially opposite the convex curved portion 330a. The first concave portion 330b may extend or substantially extend from the top of the specimen collection portion 310 to an intersection portion 335 and the second concave curved portion 330c may extend or substantially extend from the intersection portion 335 to the bottom of the base portion 305.

In some embodiments, the circumference of the specimen collection portion 310 may be greater than the circumference of the base portion 305. In some embodiments, the circumference of the specimen collection portion 310 may increase with distance from the base portion 305, for example, such that the specimen collection portion flares out from the base portion. In some embodiments, a vertical line extending from a center of a bottom of the base portion 305 up through the specimen collection portion 310 may be offset from a superior lip of the specimen collection system 300 to allow for instruments to be inserted to the bottom of the specimen collection system without difficulty.

In some embodiments, the base portion 305 may include a bottom 320 that may be coupled to a collection container, including, without limitation, conventional collection containers. For example, the bottom 320 may be coupled to a collection container through a threaded fit, a friction fit, or the like. In some embodiments, the collection system 200 may include one or more closure elements 315 configured to seal or otherwise cover the opening of the specimen collection portion 310. In some embodiments, the closure element 315 may be configured as a cap 315 formed to be coupled to collection system 300, for example, to the specimen collection portion 210 thereof. The cap 315 may be coupled to the collection system 200 through a snap fit, a threaded (screw) fit, or any other type of coupling mechanism known to those having ordinary skill in the art. In some embodiments, the cap 315 may be configured as a lid formed to be coupled to the collection system 300 through a snap- or friction-fit. In some embodiments, the closure element 315 may be tethered to the collection system 300.

In some embodiments the base portion 305 may have a width of about 3 centimeters, about 4 centimeters, about 5 centimeters, about 6 centimeters, about 8 centimeters, about 10 centimeters, or any range or value between any two of these values (including endpoints). In some embodiments the base portion 305 may have a height of about 3 centimeters, about 4 centimeters, about 5 centimeters, about 6 centimeters, about 8 centimeters, about 10 centimeters, or any range or value between any two of these values (including endpoints). In some embodiments the specimen collection portion 310 may have a width of about 3 centimeters, about 4 centimeters, about 5 centimeters, about 6 centimeters, about 8 centimeters, about 10 centimeters, about 20 centimeters, or any range or value between any two of these values (including endpoints). In some embodiments the collection system 300 may have a height of about 3 centimeters, about 4 centimeters, about 5 centimeters, about 6 centimeters, about 8 centimeters, about 10 centimeters, about 15 centimeters, about 20 centimeters, or any range or value between any two of these values (including endpoints). In some embodiments, the opening 325 may have a diameter of about 3 centimeters, about 4 centimeters, about 5 centimeters, about 6 centimeters, about 7 centimeters, about 8 centimeters, about 10 centimeters, about 15 centimeters, about 20 centimeters, or any range or value between any two of these values (including endpoints).

It will be appreciated that various of the above-disclosed and other features and functions, or alternatives thereof, may be desirably combined into many other different systems or applications. It will also be appreciated that various presently unforeseen or unanticipated alternatives, modifications, variations or improvements therein may be subsequently made by those skilled in the art which alternatives, variations and improvements are also intended to be encompassed by some embodiments described herein.

What is claimed is:

1. A medical specimen collection system, comprising:
a collection container,
a specimen collection portion in fluid communication with the collection container, the specimen collection portion being configured to receive a fluid medical specimen from a patient and to facilitate a flow of the fluid medical specimen into the specimen container, wherein the specimen collection portion has a width of about 5 centimeters to about 10 centimeters and a height of about 5 centimeters to about 10 centimeters.

2. The medical specimen collection system of claim 1, wherein the specimen collection portion is in fluid communication with a conduit configured to direct the fluid medical specimen into the specimen container.

3. The medical specimen collection system of claim 1, wherein the specimen collection portion is configured to be removably coupled to the specimen container.

4. The medical specimen collection system of claim 1, wherein the specimen collection portion comprises an inner surface having a surface area of about 5 centimeters² to about 20 centimeters².

5. The medical specimen collection system of claim 1, wherein at least a portion of the specimen collection portion is formed from at least one of a polystyrene material, a polyethylene material, a latex material, a rubber material, and a polytetrafluoroethylene material.

6. The medical specimen collection system of claim 1, wherein the specimen collection portion is flexible, thereby allowing a patient to flex the specimen collection portion into a desired position.

7. The medical specimen collection system of claim 1, further comprising at least one volume loss prevention element configured to prevent a loss of volume of the fluid medical specimen.

8. The medical specimen collection system of claim 7, wherein the at least one volume loss prevention element comprises a removable coating arranged on at least a portion of an inner surface of the specimen collection portion.

9. The medical specimen collection system of claim 1, wherein the fluid medical specimen is at least one of semen and urine.

10. A medical specimen collection system, comprising:
a base portion and a specimen collection portion extending from the base portion, the base portion and the specimen collection portion being a single monolithic piece and arranged to form a substantially bent cylinder shape,
wherein at least a portion of the specimen collection portion has a first diameter greater than a second diameter of the base portion and comprises an opening having a substantially elliptical shape,
wherein the base portion has a width of about 6 centimeters to about 10 centimeters, the opening has a diameter of about 6 centimeters to about 10 centimeters, and the medical specimen collection system has a height of about 8 centimeters to about 20 centimeters.

11. The medical specimen collection system of claim 10, wherein at least a portion of the specimen collection portion is formed from at least one of a polystyrene material, a polyethylene material, a latex material, a rubber material, and a polytetrafluoroethylene material.

12. The medical specimen collection system of claim 10, wherein the specimen collection portion is flexible, thereby allowing a patient to flex the specimen collection portion into a desired position.

13. The medical specimen collection system of claim 10, further comprising at least one volume loss prevention element configured to prevent a loss of volume of the fluid medical specimen.

14. The medical specimen collection system of claim 13, wherein the at least one volume loss prevention element comprises a removable coating arranged on at least a portion of an inner surface of the specimen collection portion.

15. The medical specimen collection system of claim 10, further comprising a closure element configured to seal the opening of the specimen collection portion.

16. A medical specimen collection system, comprising:
a base portion and a specimen collection portion extending from the base portion, the base portion and the specimen collection portion being a single monolithic piece,
wherein the base portion has a substantially cylindrical shape and the specimen collection portion has a substantially funnel shape with a narrow end of the specimen collection portion extending from an upper portion of the base portion,
wherein an upper portion of the specimen collection portion forms an opening having a first diameter greater than a second diameter of the base portion and comprises an opening having a substantially circular shape,
wherein the base portion has a width of about 6 centimeters to about 8 centimeters, the opening has a diameter of about 10 centimeters to about 15 centimeters, and the medical specimen collection system has a height of about 8 centimeters to about 20 centimeters.

17. The medical specimen collection system of claim 16, wherein at least a portion of the specimen collection portion is formed from at least one of a polystyrene material, a polyethylene material, a latex material, a rubber material, and a polytetrafluoroethylene material.

18. The medical specimen collection system of claim 16, further comprising at least one volume loss prevention element configured to prevent a loss of volume of the fluid medical specimen.

19. The medical specimen collection system of claim 18, wherein the at least one volume loss prevention element comprises a removable coating arranged on at least a portion of an inner surface of the specimen collection portion.

20. The medical specimen collection system of claim 19, further comprising a closure element configured to seal the opening of the specimen collection portion.
